(19) United States
(12) Patent Application Publication
(10) Pub. No.: US 2014/0079588 A1
(43) Pub. Date: Mar. 20, 2014

(54) SILVER-BASED DISINFECTANT COMPOSITION WITH REDUCED STAINING

(71) Applicant: Solutions BioMed, LLC, (US)

(72) Inventors: Daryl Tichy, Orem, UT (US); Brian G. Larson, Alpine, UT (US)

(73) Assignee: Solutions BioMed, LLC, Orem, UT (US)

(21) Appl. No.: 13/786,886

(22) Filed: Mar. 6, 2013

Related U.S. Application Data
(60) Provisional application No. 61/636,179, filed on Apr. 20, 2012.

(51) Int. Cl. A01N 59/16; A61L 2/18; A01N 31/02; A01N 25/00; A01N 59/00; A01N 37/02

(52) U.S. Cl. CPC A01N 59/16; A01N 59/00; A01N 37/02; A01N 31/02; A01N 25/00; A61L 2/18
USPC 422/28; 424/618; 424/489; 424/616

(57) ABSTRACT

The present invention is drawn to compositions, systems, and methods of disinfecting surfaces without the undesired staining often associated with silver composition. In one example, the composition includes water, a silver component including silver ions, a fluorescing compound that that emits absorbed light in the range of 300 nm to 450 nm, and a silver ion fixing agent adapted to chelate or reduce the silver ions.

### BACKGROUND

Disinfectants and sterilants, such as hard surface disinfectants and sterilants, are widely used in both domestic and professional settings. Generally, though both sterilants and disinfectants are used for the same purpose, i.e. to kill bacteria and/or viruses, etc., a sterilant composition exhibits a greater kill level compared to a disinfectant. Most applications require only disinfectant levels pathogen reduction, though some applications benefit considerably from the use of sterilants. For example, in the medical/dental industries, hard surfaces such as floors, walls, countertops, medical/dental instruments and equipment, etc., need to be very clean or even sterilized for safe patient care. Alternatively, though not strictly required, disinfection of surfaces in a home or business setting would also benefit from increased pathogen kill levels.

Exemplary of a commonly used hard surface cleaner is Lysol®. Though Lysol® is effective for many applications, it is not typically as effective at reducing levels of bacteria as commercially available glutaraldehyde aqueous solutions. Glutaraldehyde aqueous solutions are widely used as disinfectants and are commonly available in 1 wt % and 2 wt % solutions, particularly in medical and dental settings. Glutaraldehyde solutions are typically used for more delicate medical/dental instruments that would otherwise be susceptible to damage by other sterilization methods, e.g., autoclaving. However, glutaraldehyde is also a powerful irritant and respiratory sensitizer.

Alternative disinfectant compositions that can be safe and effective for use sometimes contain silver. Silver can have good disinfectant properties, but in some systems, particularly when used on surfaces that are plastic, glass, fabric, etc., the presence of the silver can leave an undesirable stain on the surface. Thus, alleviating the staining properties of silver in such compositions would be an advancement in the art.

### SUMMARY

In accordance with this, the present disclosure is drawn to compositions, systems, and methods of disinfecting surfaces. In one example, the present disclosure is drawn to a disinfectant composition, comprising water, a silver component including silver ions, a fluorescing compound that emits absorbed light in the range of 300 nm to 450 nm, a silver ion fixing agent adapted to chelate or reduce the silver ions. In one specific embodiment, a method of disinfecting a surface can include contacting the disinfectant composition to the surface for a sufficient period of time to disinfect the surface.

In another example, a two-part disinfectant system can comprise a first container containing Part A of a two-part solution, Part A comprising a silver component including silver ions; and a second container containing Part B of the two-part solution, Part B including water and a peroxygen. Furthermore, a fluorescing compound that emits absorbed light in the range of 300 nm to 450 nm can be admixed with at least of Part A or Part B, and a silver ion fixing agent adapted to chelate or reduce the silver ions can be admixed with at least one of Part A or Part B. Thus, upon combining Part A and Part B, a reacting disinfectant composition is formed. In one specific embodiment, a method of disinfecting a surface can comprise obtaining the two-part disinfectant system and combining Part A and Part B to form a reacting formulation. The method can further comprise contacting the reacting formulation to the surface for a sufficient period of time to disinfect the surface.

Additional features and advantages of the invention will be apparent from the detailed description that follows, which illustrates, by way of example, features of the invention.

### DETAILED DESCRIPTION

Reference will now be made to the exemplary embodiments, and specific language will be used herein to describe the same. It will nevertheless be understood that no limitation of the scope of the invention is thereby intended. Alterations and further modifications of the inventive features illustrated herein which would occur to one skilled in the relevant art and having possession of this disclosure are to be considered within the scope of the invention. It is also to be understood that the terminology used herein is used for the purpose of describing particular embodiments only. The terms are not intended to be limiting unless specified as such.

It is noted that, as used in this specification and the appended claims, the singular forms “a,” “an,” and “the” include plural referents unless the content clearly dictates otherwise.

The use of the term “disinfect,” “disinfecting,” “disinfection,” or the like is used to include not only fighting infection of virus, bacteria, fungus, or other living organisms that may contaminate an item, e.g. hard surface, medical or dental instrument, lab equipment, body surfaces, etc., but also includes disinfection of surfaces that are exposed to harmful or irritating chemicals that may be used in various clinical, laboratory, office, home, or other environments. Complete sterilization is not required for disinfection to occur. Generally, though disinfectant solutions are used for the same purpose, i.e. to kill bacteria and/or viruses, etc., a sterilant composition typically exhibits a greater kill level compared to a disinfectant. That being stated, most applications require only disinfectant levels of pathogen reduction, though other applications benefit considerably from the use of sterilants. For convenience, in the present application the term “disinfectant” is used generally to refer to both disinfectants and sterilants unless the context clearly dictates otherwise. In other words, disinfectant fluids or compositions taught herein can also be sterilants, depending on their activity against pathogens. Thus, in each instance the term “disinfectant” or the like is used, that term should be read to be inclusive of the term “sterilant” or other similar variant of this term, i.e. at least a disinfectant. However, if the term “sterilant,” “sterilization,” “sterilize” or the like is used, it is intended to mean greater level of kill associated with this term that is greater than that provided by a “disinfectant.”

The terms “solution,” “composition” and “formulation” are also used throughout the specification to describe the compositions of the present disclosure. However, as these “solutions” sometimes include colloidal transition metals or other solids, these compositions can also be described as dispersions or suspensions. As the continuous phase is typically a solution, and the transition metal can be present in ionic and/or colloidal form (and typically in small amounts and sizes), for convenience, these compositions will typically be referred to as “solutions,” “compositions” or “formulations” interchangeably. Further, sometimes a solution is referred to as a “resultant” solution or composition. This is to provide added clarity that the solution is a product of the mixing of a two-part system. As a result, the terms “solution” and “resultant solution” can be used interchangeably herein as made clear from the context of the discussion.

The term “reacting formulation” refers to compositions that are not at equilibrium, and in fact, often are actively reacting. For example, upon admixing a two-part formulation of the present disclosure, components form a reacting admixture that takes some time to come to equilibrium. During this reactive state after bringing the two-parts together, the resultant reacting formulation is more highly active for disinfecting or sterilizing surfaces in accordance with certain embodiments of the present disclosure. Once equilibrium is reached, the formulation is not as effective of a disinfectant as it is while it is actively reacting, but still can be somewhat effective, depending on the specific formulation.

As used herein, the term “colloidal” metal refers to metal particles that are in their elemental state and does not include salts or complexes. Ionic metals may also be present when colloidal metals are present, but when referring to colloidal metals, it is understood to include at least a portion of the metal in its elemental form. Alloys are considered to be in their elemental form.

When referring to the term “alloy,” it is understood that individual colloidal or metallic particles can be in the form of composites of multiple metals, or alloys can also include co-dispersions of multiple elemental metals as separate particles.

The term “peroxygen” refers to any compound containing a dioxygen (O-O) bond. Dioxygen bonds, particularly bivalent O-O bonds, are readily cleavable thereby allowing compounds containing them to act as powerful oxidizers. Non-limiting examples of classes of peroxygen compounds include peracids, peracid salts, and peroxides, such as hydrogen peroxide.

The term “two-part” when referring to the systems of the present disclosure is not limited to systems having only two parts. For example, the system can be a concentrate, and thus, is actually a three-part system, e.g., a first part including silver, a second part including a peroxygen and water, and a third part of a diluting solvent for diluting the first part, the second part, and/or the resultant solution. Either the first part or the second part, or both, can include other ingredients, such as fluorescing compounds, chelating agents, and/or reducing agents. Non-limiting examples of diluting solvents include water, alcohols, or combinations thereof. When the diluting solvent is an alcohol, it can, but need not be the same alcohol or mixture of alcohols that might already be present in the first and/or second “part” of the system. Thus, “two-part’ is specifically defined herein to mean, at least two-parts, unless the context dictates otherwise. Also, when referring to “Part A” or “Part B,” it is noted that the letter “A” or “B” is used merely for convenience, and does not infer which other co-ingredients may be present in a specific Part A or Part B formulation. Thus, “A” and “B” shall be interpreted to have no specific inference other than to identify an ingredient is from “this” part or the “other” part.

The term “container” refers to traditional containers such as tubes, dispensers, bottles, sprayers, etc. However, this term is to be viewed to be viewed more broadly to include fabrics (wipes), bandages, wrappings (foil, paper, etc.). Thus, anything capable of “containing” a fluid in accordance with embodiments of the present disclosure can be considered a container.

Concentrations, dimensions, amounts, and other numerical data may be presented herein in a range format. It is to be understood that such range format is used merely for convenience and brevity and should be interpreted flexibly to include not only the numerical values explicitly recited as the limits of the range, but also to include all the individual numerical values or sub-ranges encompassed within that range as if each numerical value and sub-range is explicitly recited. For example, a weight ratio range of about 1 wt % to about 20 wt % should be interpreted to include not only the explicitly recited limits of 1 wt % and about 20 wt %, but also to include individual weights such as 2 wt %, 11 wt %, 14 wt %, and sub-ranges such as 10 wt % to 20 wt %, 5 wt % to 15 wt %, etc.

It is noted that when a range or value is given with respect to weight percent (wt %), in the context of two-part systems, the weight percent that is referred to is that in the resultant composition or formulation after the two-part system is brought together unless clearly stated otherwise. Thus, if it is stated that a compound or compositional component is present in a formulation at from 3 wt % to 8 wt %, that indicates that the final composition that is applied to the surface is present within that weight ratio range. This is primarily applicable to the two-part embodiments described herein, as one-part systems would not create any confusion as to the applicable weight ratio range. Thus, it is understood that any ingredient present in one of the two-parts may be greater than that in the resultant formulation, and may be outside of the range described in the composition that will ultimately be applied to a given surface. Alternatively, in some instances, a weight percentage will be given and clearly labeled as being a weight percentage of one specific part of a two-part system (Part A or Part B), e.g., see certain Examples. In those instances, the weight percentages shall be as indicated. Thus, in these “two-part” embodiments, it is notable that the concentrations of each ingredient can be described in the context of concentration in the first or second composition (when specifically indicated), or the resultant solution or composition (as a default). The concentration of a compound in the first or second liquid composition will usually be lower in the resultant composition or solution than in the first or second liquid composition, as the amount typically gets diluted by the other part of the system. That being stated, this is not always the case, depending on the ingredients in the other portion of the two-part system. For example, if an ingredient is generated by a reaction, the amount may actually increase when the two-part system is combined to form the resultant composition, e.g., peracid and peroxide chemistry.

With this in mind, the present disclosure is drawn to compositions, systems, and methods of disinfecting surfaces. In one example, the present disclosure is drawn to a disinfectant composition, comprising water, a silver component including silver ions, a fluorescing compound that emits absorbed light in the range of 300 nm to 450 nm, a silver ion fixing agent adapted to chelate or reduce the silver ions. In one specific embodiment, a method of disinfecting a surface can contacting the disinfectant composition to the surface for a sufficient period of time to disinfect the surface. The method can also comprise removing the disinfectant composition from the surface after the surface is disinfected, but before any noticeable staining occurs on the surface.

In another example, a two-part disinfectant system can comprise a first container containing Part A of a two-part solution, Part A comprising a silver component including silver ions; and a second container containing Part B of the two-part solution, Part B including water and a peroxygen. Furthermore, a fluorescing compound that emits absorbed light in the range of 300 nm to 450 nm can be admixed with at least of Part A or Part B, and a silver ion fixing agent adapted to chelate or reduce the silver ions can be admixed with at least one of Part A or Part B. Thus, upon combining Part A and Part B, a reacting disinfectant composition is formed. In one specific embodiment, a method of disinfecting a surface can comprise obtaining the two-part disinfectant system and combining Part A and Part B to form a reacting formulation. The method can further comprise contacting the reacting formulation to the surface for a sufficient period of time to disinfect the surface. The method can also comprise removing the reacting formulation from the surface after the surface is disinfected, but before any noticeable staining occurs on the surface.

In some embodiments, additional additives can be present, such as peroxygens (e.g., peroxides and/or peracids), alcohols, organic acids (e.g., citric acid), a surfactant, a hypochlorites (e.g., bleach or calcium hypochlorite), benzalkonium halides, aldehydes, chlorine-based disinfectants, bromine-based disinfectants, iodophore-based disinfectants, phenolic-based decontaminants, quaternary ammonium-based disinfectants, metal salts other than silver salts, or combinations thereof. It is noted that these additional additives, even though they are listed together for convenience, are not considered to be equivalent to one another. Thus, each additive (either here or elsewhere in the present disclosure) is provided as a possible additive for a separate and distinct purpose. For example, some additives may be provided for stability of the silver, and others may contribute to disinfection properties. Any combination of these additives, or single use of these additives in the formulations of the present disclosure, is to be considered fully disclosed as if they were listed individually.

In each of the various embodiments herein, whether discussing the compositions, systems, or methods, there may be some common features of each of these embodiments that further characterize options in accordance with principles discussed herein. Thus, discussions of the compositions, systems, or methods alone are also applicable to the other embodiments not specifically mentioned.

There are several advantages of the embodiments described herein. Primarily, the compositions of the present disclosure provide effective kill levels against various types of simple pathogens, including bacterial, viral, or fungal pathogens, and in some embodiments, with more complex pathogens, such as bacterial endospores and other more complex organisms. However, though these compositions are effective for disinfecting surfaces, the silver component can leave a stain on some surfaces, including plastics, glass, wood, fabric, metal, etc. The addition of the additives described herein, e.g., fluorescing component and silver ion fixing agent, provides the benefit of not significantly (if at all) interfering with the pathogen killing properties of the formulation, while at the same time, reducing the staining properties of the silver in the formulations of the present disclosure. More specifically, the stain left by the silver in these formulations is the result of a chemical reaction or interaction between the disinfectant composition and the surface that is being cleaned, creating a permanent and objectionable color change on the surface, be it plastic, glass, fabric, metal etc. By including a fluorescing compound and a silver ion fixing agent at appropriate concentrations, the composition as applied can be converted to a non-staining composition, e.g., in some examples, a powder-like residual material that can be more easily (and often fully) removed from the surface. The removal process can be as simple as the application of tap water along with a wiping action with a paper or cloth towel. Furthermore, the fluorescing compound and the silver ion fixing agent can be included at a relatively small quantity, and do not of themselves have an objectionable staining color. Methyl salicylate, for example, provides for a light whitish powder color as a residual composition that is easily removed. Other fluorescing compounds and silver ion fixing agents may have other residual properties, but in small amounts, are much less objectionable than the staining that can occur by application of silver ions to certain surfaces.

In further detail, silver is particularly in condition for staining or development when exposed to ambient light, but especially when the light includes the spectrum from ultraviolet to blue, e.g., 300 nm to 450 nm is exemplary. As a result, a composition that inhibits this development of silver can reduce the staining power of the silver composition. It has been discovered that various fluorescing compounds and silver ion fixing agents inhibit the development properties of silver in the compositions of the present disclosure. Without being bound by any particular theory, it is believed that the fluorescing compounds luminescence to some degree in this light spectrum range where silver can be developed. By placing such chemistries in the compositions of the present disclosure, the countering of especially ultraviolet and/or blue light from ambient light sources reduces the staining effect of the disinfectant composition as a whole. Furthermore, silver ion fixing agents can also be used to either chelate with the silver ion, thus inactivating it or making it unavailable for color development, or to reduce the silver ion in solution to counteract its development/staining properties.

Though specific ingredients are described herein in detail, it is noted that there will also typically be an aqueous vehicle that includes water and optionally other ingredients, such as organic co-solvents, surfactants, and the like, so long as the additional ingredients are compatible with the compositions, systems, and methods of disinfection or sterilization described herein.

In one specific example, the composition of the present disclosure can be prepared by admixing the ingredients together and shipped for use while the ingredients remain active. In another example, the composition can be prepared by admixing at least two-parts together in accordance with a preliminary step of admixing a first liquid composition and a second liquid composition to form the composition suitable for surface disinfection or sterilization. The first liquid composition (or Part A) can comprise the silver component and the second liquid composition (or Part B) can comprise the peroxygen. The alky or aryl salicylate can be present in one or both of Part A and/or Part B, and optionally, alcohol or other ingredients can be in one or both parts. Alternatively, the first liquid composition (Part A) can comprise the peroxygen and the second liquid composition (Part B) can comprise the silver component. Thus, it is not significant what ingredients are in Part A and what ingredients are in Part B, provided the parts that are reactive or interactive with one another are kept separate, e.g., silver component separated from the peroxygen in two-part embodiments. In some instances, it may be beneficial that the two-part system actually include three-parts if there are three ingredients that should be kept separate until just prior to use. Thus, the term “two-part” should be interpreted herein to mean at least two-parts.

It is notable that when a two-part solution is brought together, reactions occur that can also reduce or increase relative concentrations of given ingredients, e.g., in the case of peracid/peroxide compositions, the peroxide component of the peracid is rapidly converted into water and oxygen within minutes of activation, and ceases to exist in some cases. Additionally, such two-part embodiments can sometimes provide effective activation for a period of weeks, e.g., up to 60 days after activation or more, depending on the specific composition. Furthermore, whether two-part system or a single solution composition, these compositions can be prepared so that they are non-corrosive or non-toxic, and emit no emissions into the environment. Furthermore, these solutions can be prepared so that they pose no health or safety issues, since all of the ingredients are essentially food grade after activation. For example, in the case of some two-part systems of peracids and peroxides, e.g., peroxyacetic acid and hydrogen peroxide, after activation by bringing the two-parts together, the dramatically altered chemical form of the peracid post-activation is no longer corrosive, exhibits no oral or inhalation toxicities, no dermal toxicities, and only mild irritation when sprayed directly into the eyes (no permanent damage to the eyes).

Turning to the compositional components more specifically, regarding the silver, a concentration in the range of 0.0001 ppm to 50,000 ppm by weight can be used and/or modified as described previously. The silver can be in ionic form (e.g. disassociate metal salt, metal ions from elemental metal, etc.), in elemental colloidal form, or in the form of a metal alloy (silver and another metal). In one specific embodiment, the silver can be in a sub-micron form (i.e. dispersion of less than 1 μm metal colloidal particles). However, larger colloidal silver particles can also be used in certain applications. It is recognized that metals will typically be oxidized to the corresponding cation in the presence of a peroxygen. With colloidal silver, for example, the surface is usually susceptible to such oxidation. Further, when colloidal silver is dispersed in a colloidal solution, there is often an amount of the metal in ionic or salt form that is also present in the suspension solution. Thus, colloidal silver may include a certain percentage of silver salt or ionic silver in solution, e.g., 10 wt % to 90 wt % of metal content can be ionic based on the total metal content.

It is also noted that any of these embodiments can also benefit from the use of alloys. For example, certain combinations of metals in an alloy may provide benefits that are related more to other consideration, such as solution stability, effectiveness on a substrate to be cleaned, etc. Examples of transition metal alloys for use in the present disclosure include, but are not limited to, copper-silver alloys, silver-manganese alloys, chromium-silver alloys, gold-silver alloys, magnesium-silver alloys, zinc-silver alloys, silver-germanium alloys, and the like.

Exemplary colloidal silvers that can be used include those sold by Solutions IE, Inc. under the trade names CS Plus and CS Ultra. Other colloidal silver products that can be used as the silver source include ASAP, Sovereign Silver, Silver Max, or the like. In one embodiment, the colloidal silver particles used in the present disclosure can have a particle size range of from 0.001 μm to 1.0 μm. In another embodiment, the colloidal silver particles can have a size range of from 0.030 μm to 0.5 μm. In still another embodiment, the average particle size can be 0.35 μm to 0.45 μm. If used in ionic form, silver salts can include, but are not limited to silver nitrate, silver acetate, silver citrate, silver oxide, and/or silver carbonate.

Turning to the fluorescing compound, typically, compounds are selected that emit absorbed light in the range of 300 nm to 450 nm, as this is the range that seems to be the most problematic for development or color change of silver ions in solution. Exemplary compounds effective for this purpose include distyrylbiphenyls, stilbenes, coumarins, benzothiazoles, quinines, and/or alkyl or aryl salicylates. In one example, the fluorescing compound can comprise C₁-C₅ alkyl salicylate, such as methyl salicylate, ethyl salicylate, or amyl salicylate. In another embodiment, the fluorescing compound can comprise an aryl salicylate, such as benzyl salicylate. A specific example of a coumarin that can be used is 7-hydroxycoumarin, though others are also effective. An example of a distyrylbiphenyl compound that can be used is disulfonic distyrylbiphenyl biphenyl. Likewise, and example of a quinine that can be selected for use is quinine sulfate. With these specific compounds listed, it is understood that other compounds can also be used that provide the function of emitting absorbed light in the range of 300 nm to 450 nm. Thus, the fluorescing compound can act like sun screens or sun blocks to prevent light from decolorizing or staining surfaces with compositions that contain silver ions. Also, it is also noted that in selecting fluorescing compounds for use, it is desirable to select compounds that are effective for reducing the staining properties of silver ions on various surfaces, but which do not of themselves provide an objectionable stain would be more objectionable than that inherently caused by the silver ions on the surface.

Regarding the silver ion fixing agent, there are essentially two types of silver ion fixing agents that are notable (one or both can be used as the silver ion fixing agent), namely chelating agents and reducing agents. Chelation, by definition, occurs when an organic ligand forms a ring structure with a metal ion (silver in this case), with the metal ion acting as the closing member. Typically, chelates are 5- or 6-membered rings with 4- or 7-membered rings being less common or less stable. Chelation is useful in minimizing the staining properties of silver because the organic ligand acts to tie up the metal ion in solution, converting it to a more chemically inert form that is much less light sensitive. Examples of chelates that can be used include oxalates such as sodium oxylate, oxalic acid, salicylates such as C₁-C⁵ alkyl or aryl salicyaltes, acetylsalicylates, acetylsalicylic acid, malonic acid, malonates such as sodium malonate, ethylenediaminetetraacetic acids such as disodium EDTA, citric acid, citrates such as sodium citrate, or amino acids that are at least 0.1 wt % water soluble. The other class of silver ion fixing agents that can be used include reducing agents that can modify or stabilize silver ions in the compositions of the present disclosure. Examples include oxalic acid, oxalates such as sodium oxylate, ascorbic acid, sulfites such as sodium sulfite, or phosphites.

It is noted that some compounds act as both a chelator and a reducer, and thus, the list has some overlap. Furthermore, it is also noted that some silver ion fixing agents also are functional as fluorescing compounds in accordance with examples of the present disclosure. However, when a compound is listed as both a fluorescing compound and a silver ion fixing agent, it is understood that two compounds be selected for use in accordance with examples of the present disclosure, i.e. one which functions at least as a fluorescing compound and one that functions at least as a silver ion fixing agent.

It is also noted that it can be desirable to use RO water as the suspension medium for the colloidal and/or ionic silver that is mixed with the other ingredients. In a more detailed aspect, the RO water can also be distilled, resulting in 18-20 MΩ water, though this is not required.

Regarding certain optional ingredients that can be used in accordance with examples of the present disclosure, peroxygens can also be effective to be used in conjunction with the compositions or 2-part systems described herein. For example, this component can be present in the compositions of the present disclosure at from 0.0001 wt % to 25 wt %, with the upper end of the range being modifiable as described below herein. The peroxygen can be a single compound or a combination of multiple peroxygen compounds or peroxygen forming compounds. In one embodiment, the peroxygen can be any aliphatic or aromatic peracid (or peroxyacid) that is functional for disinfection purposes in accordance with embodiments of the present disclosure. While any functional peroxyacid can be used, peroxyacids containing from 1 to 7 carbons are the most practical for use. These peroxyacids can include, but not be limited to, peroxyformic acid, peroxyacetic acid, peroxyoxalic acid, peroxypropanoic acid, perlactic acid, peroxybutanoic acid, peroxypentanoic acid, peroxyhexanoic acid, peroxyadipic acid, peroxycitric, and/or peroxybenzoic acid. The peroxyacid used in the present disclosure can be prepared using any method known in the art. When the peroxyacid is prepared from an acid and hydrogen peroxide, the resultant mixture contains both the peroxyacid and the corresponding acid that it is prepared from. For example, in embodiments that utilize peroxyacetic acid, the presence of the related acid (acetic acid) provides stability to the mixture, as the reaction is an equilibrium between the acid, hydrogen peroxide, and the peroxyacid and water, as follows:

H₂O₂+CH₃COOH [symbol] CH₃COO—OH+H₂O

Peracid salts, such as salts of the above listed peracids, can also be included as the peroxygen component of the solutions. Non-limiting examples of such salts include permanganates, perborates, perchlorates, peracetates, percarbonates, persulphates, and the like. The salts can be used alone or in combination with each other or other peroxygen compounds to form the peroxygen component of the disclosure.

In another embodiment, the peroxygen component of the disclosure can include a peroxide compound. While hydrogen peroxide is considered to be a desirable peroxide for use in accordance with embodiments of the present disclosure, other peroxides can also be used, such as metal peroxides and peroxyhydrates. The metal peroxides that can be used include, but are not limited to, sodium peroxide, magnesium peroxide, calcium peroxide, barium peroxide, and/or strontium peroxide. Other salts (for example sodium percarbonate) have hydrogen peroxide associated therewith much like waters of hydration, and these could also be considered to be a source of hydrogen peroxide, thereby producing hydrogen peroxide in situ. As mentioned above, the peroxides can be used alone or in combination with other peroxygen compounds to form the peroxygen component of the present disclosure.

If an alcohol is present in the composition, or in one or both of Part A and Part B of the two-part system, in one example, the alcohol can be present (in the composition or resultant composition from the two-part system) at from about 0.0001 wt % to 95 wt %, with the upper end and lower end of the range being modifiable as described hereinafter. Examples of alcohols that can be used include, but are limited to, aliphatic alcohols and other carbon-containing alcohols, having from 1 to 24 carbons (C₁-C₂₄ alcohol). It is to be noted that “C₁-C₂₄ alcohol” does not necessarily imply only straight chain saturated aliphatic alcohols, as other carbon-containing alcohols can also be used within this definition, including branched aliphatic alcohols, alicyclic alcohols, aromatic alcohols, unsaturated alcohols, as well as substituted aliphatic, alicyclic, aromatic, and unsaturated alcohols, etc. In one embodiment, the aliphatic alcohols can be C₁ to C₅ alcohols including methanol, ethanol, propanol and isopropanol, butanols, and pentanols, due to their availability and lower boiling points. This being stated, polyhydric alcohols can also be used effectively in accordance with the present disclosure. Examples of polyhydric alcohols which can be used in the present disclosure include but are not limited to ethylene glycol (ethane-1,2-diol), glycerin (or glycerol, propane-1,2,3-triol), sorbitol, and propane-1,2-diol. Other non-aliphatic alcohols may also be used including but not limited to phenols and substituted phenols, erucyl alcohol, ricinolyl alcohol, arachidyl alcohol, capryl alcohol, capric alcohol, yl alcohol, lauryl alcohol (1-dodecanol), myristyl alcohol (1-tetradecanol), cetyl (or palmityl) alcohol (1-hexadecanol), stearyl alcohol (1-octadecanol), isostearyl alcohol, oleyl alcohol (cis-9-octadecen-1-ol), palmitoleyl alcohol, linoleyl alcohol (9Z, 12Z-octadecadien-1-ol), elaidyl alcohol (9E-octadecen-1-ol), elaidolinoleyl alcohol (9E, 12E-octadecadien-1-ol), linolenyl alcohol (9Z, 12Z, 15Z-octadecatrien-1-ol), elaidolinolenyl alcohol (9E, 12E, 15-E-octadecatrien-1-ol), combinations thereof, and the like.

In some embodiments, for practical considerations, methanol, ethanol, propanols, butanols, pentanols, and denatured alcohols (mixtures of ethanol and smaller amounts of methanol and other possible minor amounts of organics) can often be used because of their availability and cost. Glycerol or sorbitol can also be used in some embodiments. Since the desire is typically to provide a highly effective disinfectant compositions or two-part systems, then alcohols can be selected that satisfy this desire. When considering the amount of alcohol to use, one skilled in the art can stay within the above-described ranges, or modify these ranges for a particular application, considering such things as whether alcohol selected for use is polyhydric, whether the alcohol is food grade or non-toxic, mixtures of alcohols, etc.

The single part compositions and two-part systems of the present disclosure can comprise the ingredients described herein at various concentrations. It is noted, however, when describing exemplary ranges, the ranges given are based on the composition as prepared, or the resultant composition once multiple parts are brought together. Thus, in one example, the composition (or two-part system after being brought together) can comprise an aqueous vehicle including water, from 0.0001 ppm to 50,000 ppm by weight of the silver including at least some silver ions, from 0.01 wt % to 5 wt % of the fluorescing compound, from 0.01 wt % to 10 wt % of the silver ion fixing agent, and optionally, other ingredients such peroxygen, alcohol, or other vehicle components. It is noted that the concentration of the silver content, including ionic and/or colloidal content, can be independently modified to 10 ppm, 1 ppm, 0.1 ppm, 0.01 ppm, 0.001 ppm by weight at the lower end of the range, and/or to 10,000 ppm, 5,000 ppm, or 1,500 ppm by weight at the upper end of the range. Likewise, the fluorescing compound can be independently modified to at the lower end of the range 0.02 wt %, 0.05 wt %, 0.1 wt %, or 1 wt %, and the upper end of the range can be independently modified to 2 wt %, 1 wt %, or 0.1 wt %, provided the lower end of the range is a lower than the upper end of the range. The silver ion fixing agent can also be independently modified to at the lower end of the range 0.02 wt %, 0.05 wt %, 0.1 wt %, or 1 wt %, and the upper end of the range can be independently modified to 5 wt %, 2 wt %, 1 wt %, 0.2 wt %, or 0.1 wt %, provided the lower end of the range is lower than the upper end of the range.

Regarding the optional ingredients that can be present in the composition or two-part system (once combined), if a peroxygen is present, as mentioned, it is typically present at from 0.0001 wt % to 25 wt %. However, it is noted that the lower end of the range of the peroxygen of the composition can be independently modified to 0.05 wt % or 0.1 wt %, and/or the upper end of the range can be independently modified to10 wt %, 5 wt %, 3 wt %, or 1.5 wt % in accordance with specific embodiments of the present disclosure. It is also noted that an alcohol can be present at from 0.0001 wt % to 95 wt %, with the lower end of the range of the alcohol independently modifiable to 0.05 wt % or 0.1 wt %, and the upper end of the range being independently modifiable to 40 wt %, 30 wt %, 20 wt % or 10 wt % in accordance with specific embodiments of the present disclosure.

As these ranges are merely exemplary, one skilled in the art could modify these ranges for a particular application, considering such things as the type of alcohol (polyhydric, mixtures, etc.); the type of peroxygen (peroxide, peracid, combination of peroxide/peracid, etc.); the type of metal (ionic, colloidal, alloy, etc.); the type of fluorescing compound; the type of silver ion fixing agent; etc. Further, it is noted that any combination of these upper and lower limits for each of the ingredients are expressly included herein.

It is noted that in some examples of the present disclosure, certain types of toxic components can be omitted from the compositions of the present disclosure. Non-food-grade ingredients which can be omitted from the compositions of the present disclosure include, but are not limited to, aldehydes such as glutaraldehyde; chlorine-based disinfectants; chlorine and bromine-based disinfectants; iodophore-based disinfectants; phenolic-based disinfectants, quaternary ammonium-based disinfectants; and/or the like.

The storage and dispensing of the compositions or the two-part systems of the present disclosure can be by any acceptable manner known in the art. Specific non-limiting examples of such systems include those adapted for dispensing of one or multiple fluids, aerosols, sprays, mists, gels, drops, washes, wipes, etc.

### EXAMPLES

The following examples illustrate the embodiments of the disclosure that are presently best known. However, it is to be understood that the following are only exemplary or illustrative of the application of the principles of the present disclosure. Numerous modifications and alternative compositions, methods, and systems may be devised by those skilled in the art without departing from the spirit and scope of the present disclosure. The appended claims are intended to cover such modifications and arrangements. Thus, while the present disclosure has been described above with particularity, the following examples provide further detail in connection with what are presently deemed to be the most practical and preferred embodiments of the disclosure.

### Example 1

### Preparation of Disinfectant Solution

An aqueous disinfectant composition is prepared in accordance with embodiments of the present disclosure, which includes the following ingredients in approximate amounts: 92 wt % distilled water containing 300 ppm by weight silver ions (silver nitrate source); 2 wt % quinine sulfate; 1 wt % gamma cyclodextrin; and 5 wt % sodium oxalate. It is noted that there will be less than 300 ppm by weight of the silver when based on the composition as a whole.

### Example 2

### Preparation of Disinfectant Solution

An aqueous disinfectant composition is prepared in accordance with embodiments of the present disclosure, which includes the following ingredients in approximate amounts: 97.7 wt % distilled water containing 200 ppm by weight colloidal silver; 0.8 wt % 7-hydroxycoumarin (umbelliferone); 1 wt % gamma cyclodextrin; and 0.5 wt % sodium citrate. It is noted that there will be less than 200 ppm by weight of the silver when based on the composition as a whole.

### Example 3

### Preparation of Disinfectant Solution

An aqueous disinfectant composition is prepared in accordance with embodiments of the present disclosure, which includes the following ingredients in approximate amounts: 99.4 wt % distilled water containing 300 ppm by weight colloidal silver; 0.5 wt % of a distyrylbiphenyl (Keyfluor CBS-X); 0.06 wt % EDTA; and 0.04 oxalic acid. It is noted that there will be less than 300 ppm by weight of the silver when based on the composition as a whole.

### Example 4

### Preparation of Disinfectant Solution

An aqueous disinfectant composition is prepared in accordance with embodiments of the present disclosure, which includes the following ingredients in approximate amounts: 85 wt % distilled water containing 600 ppm by weight colloidal silver; 0.5 wt % methyl salicylate; 0.8 wt % 7-hydroxycoumarin (umbelliferone); 1 wt % gamma cyclodextrin; 0.5 wt % sodium citrate; 8 wt % ethanol; and 4.2 wt % peroxyacetic acid. To the composition is added a small amount, i.e. <3 wt % based on the aqueous composition as a whole, of hydrogen peroxide to stabilize the peroxyacetic acid. It is noted that there will be less than 600 ppm by weight of the colloidal silver when based on the composition as a whole.

### Example 5

### Preparation of Disinfectant Solution

An aqueous disinfectant composition is prepared in accordance with embodiments of the present disclosure, which includes the following ingredients in approximate amounts: 85 wt % distilled water containing 600 ppm by weight colloidal silver; 0.05 wt % methyl salicylate; 0.5 wt % of a distyrylbiphenyl (Keyfluor CBS-X); 0.06 wt % EDTA; 0.04 wt % oxalic acid 9.45 wt % isopropanol; 5 wt % peroxypropanoic acid. To the composition is added a small amount of sodium peroxide to stabilize the peroxypropanoic acid. It is noted that there will be less than 600 ppm by weight of the colloidal silver when based on the aqueous vehicle content as a whole.

### Example 6

### Preparation of Disinfectant Solution

An aqueous disinfectant composition is prepared in accordance with embodiments of the present disclosure, which includes the following ingredients in approximate amounts: 75 wt % RO water (reverse osmosis water) containing 1,500 ppm by weight colloidal silver; 1.5 wt % 7-hydroxycoumarin (umbelliferone); 2 wt % oxalic acid; 2 wt % sodium citrate; 14.5 wt % ethanol; and 5 wt % peroxyacetic acid. To the composition is added a small amount of hydrogen peroxide and acetic acid to the solution to stabilize the peracetic acid. It is noted that there will be less than 1500 ppm by weight of the colloidal silver when based on the aqueous vehicle content as a whole.

### Example 7

### Preparation of Disinfectant Solution

An aqueous disinfectant composition is prepared in accordance with embodiments of the present disclosure, which includes the following ingredients in approximate amounts: 85 wt % distilled water containing 10,000 ppm by weight colloidal silver; 5 wt % methyl salicylate; 5 wt % gamma cyclodextrin; and 5 wt % sodium citrate. It is noted that there will be less than 10,000 ppm by weight of the colloidal silver when based on the aqueous vehicle content as a whole.

### Example 8

### Preparation of Disinfectant Solution

An aqueous disinfectant composition is prepared in accordance with embodiments of the present disclosure, which includes the following ingredients in approximate amounts: 85 wt % distilled water containing 80 ppm by weight colloidal silver; 1 wt % quinine sulfate; 9 wt % ethanol; 2 wt % acetylsalicylate; 3 wt % malonic acid. It is noted that there will be less than 80 ppm by weight of the colloidal silver when based on the aqueous vehicle content as a whole.

### Example 9

### Preparation of Disinfectant Solution

An aqueous disinfectant composition is prepared in accordance with embodiments of the present invention, which includes the following ingredients in approximate amounts: 5 wt % isopropanol; 1.3 wt % peroxypropanoic acid (from a 6 wt % solution); less than 3 wt % of a peroxide, e.g., sodium peroxide, to stabilize the peroxypropanoic acid; 0.05 wt % ethyl salicylate; 2 wt % of a distyrylbiphenyl (Keyfluor CBS-X); 8 wt % sodium oxylate; and the balance being water containing 600 ppm ionic silver (from a silver salt source such as silver nitrate, silver chloride, etc.). It is noted that there will be less than 600 ppm by weight of the ionic silver when based on the aqueous vehicle content as a whole.

### Example 10

### Preparation of Disinfectant Solution Using Two-part System

A two-part disinfectant system is provided. The first liquid composition of the system includes a solution of 20 parts by weight glycerol, 29.6 parts by weight water, and 0.03 parts by weight methyl salicylate, 0.04 parts by weight oxalic acid, 0.3 parts by weight sodium citrate, and 0.03 parts by weight colloidal silver (600 ppm). The second liquid composition includes 1.3 parts by weight peracetic acid and 48.7 parts by weight water. The two components are kept separate until immediately before the disinfectant is desired for use. The disinfectant solution is made by mixing the two components at about a 1:1 (first:second) weight ratio. In this embodiment, less than 3 wt % of hydrogen peroxide can optionally be added to further stabilize the system. This disinfectant solution can be used effectively to disinfect and sterilize a variety of surfaces.

### Example 11

### Preparation of Disinfectant Solution Using Two-part System

A two-part disinfectant system is provided. The first liquid composition of the system includes a solution of about 8.7 parts by weight glycerol, about 0.8 parts 7-hydroxycoumarin (umbelliferone), about 1 part by weight gamma cyclodextrin, about 0.5 parts by weight sodium citrate, and about 80 parts by weight of a silver hydrosol (200 ppm colloidal silver). The second liquid composition of the system is includes an aqueous solution of 15 wt % peracetic acid in water. The two components are kept separate until immediately before the disinfectant is desired for use. The two components are combined at a weight ratio of 91:9 (first:second). This disinfectant solution can be used effectively to disinfect and sterilize a variety of surfaces. It is noted that there will be slightly less than 200 ppm by weight of the colloidal silver when based on the resultant disinfectant composition as a whole. This disinfectant solution can be used effectively to disinfect and sterilize a variety of surfaces.

### Example 12

### Preparation of Disinfectant or Sterilant Solution Using Two-part System

A two-part disinfectant or sterilant system is provided. The liquid composition of the system includes a solution of about 9.4 parts by weight glycerol, about 0.5 parts by weight of a distyrylbiphenyl (Keyfluor CBS-X), about 0.06 parts by weight EDTA, about 0.04 parts by weight oxalic acid, and about 87 parts by weight of a silver hydrosol (300 ppm colloidal silver). The second liquid composition of the system is an aqueous solution of 15 wt % peracetic acid. The two components are kept separate until immediately before the disinfectant is desired for use. The disinfectant solution is made by mixing the two components at about a 97:3 (first:second) weight ratio. It is noted that there will be less than 300 ppm by weight of the colloidal silver when based on the resultant disinfectant composition as a whole. This disinfectant solution can be used effectively to disinfect and sterilize a variety of surfaces.

### Example 13

### Preparation of disinfectant or sterilant solution using two-part system

A two-part disinfectant or sterilant system is provided. The first liquid composition includes, by weight, 9 parts ethanol, 40 parts water, 0.2 parts methyl salicylate, 0.6 parts gamma cyclodextrin, 0.1 parts oxalic acid, and 0.1 parts colloidal silver (2,000 ppm). The second liquid composition includes, by weight, 1.3 parts peroxypropanoic acid and 48.7 parts water. The two components are kept separate until immediately before the disinfectant is desired for use. The disinfectant solution is made by mixing the two components at about a 1:1 (first:second) weight ratio. In this embodiment, less than 3 wt % of hydrogen peroxide can optionally be added to further stabilize the system. This disinfectant solution can be used effectively to disinfect and sterilize a variety of surfaces.

### Example 14

### Preparation of Disinfectant or Sterilant Solution Using Two-part System

A two-part disinfectant or sterilant system is provided. The first liquid composition includes, by weight, 20 parts denatured alcohol, 25.4 parts water, 1.55 parts 7-hydroxycoumarin (umbelliferone); 2 parts gamma cyclodextrin; and 1 part sodium citrate, and 0.05 parts silver and copper alloy (1,000 ppm). The second liquid composition is includes, by weight, 3 parts percitric acid and 47 parts water. The two components are kept separate until immediately before the disinfectant is desired for use. The disinfectant solution is made by mixing the two components at about a 1:1 (first:second) weight ratio. In this embodiment, less than 3 wt % of hydrogen peroxide can optionally be added to further stabilize the system. This disinfectant solution can be used effectively to disinfect and sterilize a variety of surfaces.

### Example 15

### Preparation of Disinfectant or Sterilant Solution Using Two-part System

A two-part disinfectant or sterilant system is provided. The first liquid composition included approximately 0.015 wt % silver, 0.0004 wt % sorbitol, 0.5 wt % methyl salicylate, 0.4 wt % oxalic acid, 0.25 wt % sodium citrate, 10 wt % ethanol, and the balance water. The second liquid composition is included 22 wt % hydrogen peroxide, 15 wt % peroxyacetic acid, 15 wt % acetic acid, and the balance water. The two components are kept separate until immediately before the disinfectant is desired for use, though after activation, the composition can continue to be effective for several weeks. In this example, it is noted that activation of the resultant composition occurs by pouring the entire contents of the second liquid composition containing a premeasured 37.8 mL for gallon size (10.0 mL for liter) into the first liquid composition containing a premeasured 3,747.6 mL for gallon size (990.0 mL for liter) to achieve a 99:1 mixed volume ratio, which can be followed by agitating the combined solution for 15 seconds.

### Example 16

### Preparation of Disinfectant or Sterilant Solution Using Two-part System

A two-part disinfectant or sterilant system is provided. The first liquid composition included approximately 0.03 wt % silver, 2 wt % quinine sulfate, 1 wt % gamma cyclodextrin, 5 wt % sodium oxylate, 10 wt % ethanol, and the balance water. The second liquid composition is included 22 wt % hydrogen peroxide, 15 wt % peroxyacetic acid, 15 wt % acetic acid, and the balance water. The two components are kept separate until immediately before the disinfectant is desired for use, though after activation, the composition can continue to be effective for several weeks. In this example, it is noted that activation of the resultant composition occurs by pouring the entire contents of the second liquid composition containing a premeasured 37.8 mL for gallon size (10.0 mL for liter) into the first liquid composition containing a premeasured 3,747.6 mL for gallon size (990.0 mL for liter) to achieve a 99:1 mixed volume ratio, which can be followed by agitating the combined solution for 15 seconds.

### Example 17

### Application to Various Substrates with Reduced Staining

A composition admixed together from two parts, as described in Example 15 above, can be used to test its stain ameliorating properties. For comparison purposes, a similar composition prepared without the presence of the methyl salicylate, oxalic acid, and sodium citrate was shown to cause staining on various substrates including plastics, cloth, metal, and glass. In contrast, the two part system of Example 15 (with methyl salicylate, oxalic acid, and sodium citrate) was admixed and flooded (much more than would normally be applied) on the same substrates to compare staining profiles. After application of the composition of Example 15 was applied to the various substrates, the compositions on the substrates were flooded with bright, broad-spectrum light having intensities beyond those found in typical ambient environments. This was done to verify stain reduction properties of the methyl salicylate in a composition that otherwise would stain the various substrates. Thus, excess composition and excess light was used to attempt the achieving of significant staining on the surfaces. It was observed that the composition of Example 15 significantly reduced staining compared to compositions that did not include the fluorescing compound, chelating agent, and/or reducing agent.

While the invention has been described with reference to certain preferred embodiments, those skilled in the art will appreciate that various modifications, changes, omissions, and substitutions can be made without departing from the spirit of the invention. It is therefore intended that the invention be limited only by the scope of the appended claims.

What is claimed is:

1. A disinfectant composition, comprising:
water;
a silver component including silver ions;
a fluorescing compound that emits absorbed light in the range of 300 nm to 450 nm;
a silver ion fixing agent adapted to chelate or reduce the silver ions.

2. The composition of claim 1, wherein the silver component comprises elemental colloidal silver and the silver ions in solution.

3. The composition of claim 1, wherein the elemental colloidal silver has an average particle size of from 0.030 μm to 0.5 μm.

4. The composition of claim 1, wherein the silver component comprises a silver salt and the silver ions in solution.

5. The composition of claim 1, wherein the silver component comprises an alloy of elemental silver and an elemental metal selected from the group of ruthenium, rhodium, osmium, iridium, palladium, platinum, copper, gold, manganese, zinc, and germanium.

6. The composition of claim 1, wherein the silver component is present at from 0.0001 ppm to 50,000 ppm by weight of silver content.

7. The composition of claim 1, wherein the silver component is present at from 10 ppm to 1,500 ppm by weight of silver content.

8. The composition of claim 1, wherein the fluorescing compound is selected from the group consisting of distyrylbiphenyl, stilbenes, coumarins, benzothiazoles, quinines, alkyl or aryl salicylates, and combinations thereof.

9. The composition of claim 8, wherein the fluorescing compound comprises C₁-C₅ alkyl salicylate.

10. The composition of claim 9, wherein the C₁-C₅ alkyl salicylate is ethyl salicylate or amyl salicylate.

11. The composition of claim 9, wherein the C₁-C₅ alkyl salicylate is methyl salicylate.

12. The composition of claim 8, wherein the fluorescing compound comprises an aryl salicylate.

13. The composition of claim 12, wherein the aryl salicylate is benzyl salicylate.

14. The composition of claim 8, wherein the fluorescing compound is 7-hydroxycoumarin.

15. The composition of claim 8, wherein the fluorescing compound is a disulfonic distyrylbiphenyl biphenyl.

16. The composition of claim 8, wherein the fluorescing compound is quinine sulfate.

17. The composition of claim 1, wherein the fluorescing compound is present at from 0.01 wt % 5 wt %.

18. The composition of claim 1, wherein the fluorescing compound is present at from 0.01 wt % to 1 wt %.

19. The composition of claim 1, wherein the fluorescing compound is present at from 0.02 wt % to 0.1 wt %.

20. The composition of claim 1, wherein the silver ion fixing agent is a chelating agent for the silver ions.

21. The composition of claim 1, wherein the silver ion fixing agent is a reducing agent for the silver ions.

22. The composition of claim 1, wherein the silver ion fixing agent is both a chelating agent and a reducing agent for the silver ions.

23. The composition of claim 1, wherein the silver ion fixing agent includes both a chelating agent and a separate reducing agent.

24. The composition of claim 1, wherein the silver ion fixing agent is selected from the group consisting of sodium oxalate, oxalic acid, malonic acid, acetylsalicylate, aspirin, malonic acid, sodium malonate, disodium ethylenediaminetetraacetic acid, citric acid, sodium citrate, an amino acid having at least 0.1 wt % water solubility, ascorbic acid, sulfites, phosphites, quinines, and mixtures thereof.

25. The composition of claim 1, wherein the silver ion fixing agent is present at a total concentration from 0.01 wt % to 10 wt %.

26. The composition of claim 1, wherein the silver ion fixing agent is present at a total concentration from 0.01 wt % to 1 wt %.

27. The composition of claim 1, wherein the silver ion fixing agent is present at a total concentration from 0.02 wt % to 0.2 wt %.

28. The composition of claim 1, further comprising a peroxygen.

29. The composition of claim 28, wherein the peroxygen is a peracid.

30. The composition of claim 28, wherein the peroxygen is a peroxide.

31. The composition of claim 28, wherein the peroxygen includes a peracid and a peroxide.

32. The composition of claim 28, wherein the peroxygen is present at from 0.0001 wt % to 25 wt %.

33. The composition of claim 28, wherein the peroxygen is present at from 0.05 wt % to 5.0 wt %.

34. The composition of claim 28, wherein the peroxygen is present at from 0.1 wt % to 1.5 wt %.

35. The composition of claim 1, further comprising an alcohol.

36. The composition of claim 35, wherein the alcohol includes a member selected from the group consisting of methanol, ethanol, a propanol, a butanol, a pentanol, and mixtures thereof.

37. The composition of claim 35, wherein the alcohol includes a polyhydric alcohol.

38. The composition of claim 1, adapted for application in the form of an aerosol, spray, pre-soaked fabric, gel, or foam.

39. The composition of claim 1, in the form of a dilutable concentrate.

40. The composition of claim 1, wherein the silver component is present in a form and at a concentration that causes silver staining when applied to a plastic surface, and the fluorescing compound and the silver ion fixing agent are present at concentrations that reduces the staining compared to an identical composition devoid of the fluorescing compound or the silver ion fixing agent.

41. The composition of claim 1, further comprising an organic acid different than the silver ion fixing agent, a surfactant, a hypochlorite, a benzalkonium halide, an aldehyde, a chlorine-based disinfectant, a bromine-based disinfectant, an iodophore-based disinfectant, phenolic-based disinfectant, a quaternary ammonium-based disinfectant, a metal salt disinfectant other than silver salt, or combination thereof.

42. A method of disinfecting a surface, comprising contacting the composition of claim 1 to the surface for a sufficient period of time to disinfect the surface.

43. The method of claim 42, further comprising the step of removing at least a portion of the composition from the surface after the surface is disinfected, but before any noticeable staining occurs on the surface.

44. A two-part disinfectant system, comprising:
a first container containing Part A of a two-part solution, Part A comprising a silver component including silver ions;
a second container containing Part B of the two-part solution, Part B including water and a peroxygen;
a fluorescing compound that emits absorbed light in the range of 300 nm to 450 nm admixed with at least of Part A or Part B; and
a silver ion fixing agent adapted to chelate or reduce the silver ions admixed with at least one of Part A or Part B,
wherein upon combining Part A and Part B, a reacting disinfectant composition is formed.

45. The two-part disinfectant system of claim 44, wherein the silver component comprises elemental colloidal silver and the silver ions in solution.

46. The two-part disinfectant system of claim 44, wherein the elemental colloidal silver has an average particle size of from 0.030 μm to 0.5 μm.

47. The two-part disinfectant system of claim 44, wherein the silver component comprises a silver salt and the silver ions in solution.

48. The two-part disinfectant system of claim 44, wherein the silver component comprises an alloy of elemental silver and an elemental metal selected from the group of ruthenium, rhodium, osmium, iridium, palladium, platinum, copper, gold, manganese, zinc, and germanium.

49. The two-part disinfectant system of claim 44, wherein the silver component is present in the reacting disinfectant composition at from 0.0001 ppm to 50,000 ppm by weight of silver content.

50. The two-part disinfectant system of claim 44, wherein the silver component is present in the reacting disinfectant composition at from 10 ppm to 1,500 ppm by weight of silver content.

51. The two-part disinfectant system of claim 44, wherein the fluorescing compound is selected from the group consisting of distyrylbiphenyl, stilbenes, coumarins, benzothiazoles, quinines, alkyl or aryl salicylates, and combinations thereof.

52. The two-part disinfectant system of claim 51, wherein the fluorescing compound comprises C₁-C₅ alkyl salicylate.

53. The two-part disinfectant system of claim 52, wherein the C₁-C₅ alkyl salicylate is ethyl salicylate or amyl salicylate.

54. The two-part disinfectant system of claim 52, wherein the C₁-C₅ alkyl salicylate is methyl salicylate.

55. The two-part disinfectant system of claim 51, wherein the fluorescing compound comprises an aryl salicylate.

56. The two-part disinfectant system of claim 52, wherein the aryl salicylate is benzyl salicylate.

57. The two-part disinfectant system of claim 51, wherein the fluorescing compound is 7-hydroxycoumarin.

58. The two-part disinfectant system of claim 51, wherein the fluorescing compound is a disulfonic distyrylbiphenyl biphenyl.

59. The two-part disinfectant system of claim 51, wherein the fluorescing compound is quinine sulfate.

60. The two-part disinfectant system of claim 44, wherein the fluorescing compound is present in the reacting disinfectant composition at from 0.01 wt % 5 wt %.

61. The two-part disinfectant system of claim 44, wherein the fluorescing compound is present in the reacting disinfectant composition at from 0.01 wt % to 1 wt %.

62. The two-part disinfectant system of claim 44, wherein the fluorescing compound is present in the reacting disinfectant composition at from 0.02 wt % to 0.1 wt %.

63. The two-part disinfectant system of claim 44, wherein the silver ion fixing agent is a chelating agent for the silver ions.

64. The two-part disinfectant system of claim 44, wherein the silver ion fixing agent is a reducing agent for the silver ions.

65. The two-part disinfectant system of claim 44, wherein the silver ion fixing agent is both a chelating agent and a reducing agent for the silver ions.

66. The two-part disinfectant system of claim 44, wherein the silver ion fixing agent includes both a chelating agent and a separate reducing agent.

67. The two-part disinfectant system of claim 44, wherein the silver ion fixing agent is selected from the group consisting of sodium oxalate, oxalic acid, acetylsalicylate, aspirin, malonic acid, sodium malonate, disodium ethylenediaminetetraacetic acid, citric acid, sodium citrate, an amino acid having at least 0.1 wt % water solubility, ascorbic acid, sulfites, phosphites, quinines, and mixtures thereof.

68. The two-part disinfectant system of claim 44, wherein the silver ion fixing agent is present in the reacting disinfectant composition at a total concentration from 0.01 wt % to 10 wt %.

69. The two-part disinfectant system of claim 44, wherein the silver ion fixing agent is present in the reacting disinfectant composition at a total concentration from 0.01 wt % to 1 wt %.

70. The two-part disinfectant system of claim 44, wherein the silver ion fixing agent is present in the reacting disinfectant composition at a total concentration from 0.02 wt % to 0.2 wt %.

71. The two-part disinfectant system of claim 44, further comprising a peroxygen.

72. The two-part disinfectant system of claim 71, wherein the peroxygen is a peracid.

73. The two-part disinfectant system of claim 71, wherein the peroxygen is a peroxide.

74. The two-part disinfectant system of claim 71, wherein the peroxygen includes a peracid and a peroxide.

75. The two-part disinfectant system of claim 71, wherein the peroxygen is present at from 0.0001 wt % to 25 wt %.

76. The two-part disinfectant system of claim 71, wherein the peroxygen is present at from 0.05 wt % to 5.0 wt %.

77. The two-part disinfectant system of claim 71, wherein the peroxygen is present at from 0.1 wt % to 1.5 wt %.

78. The two-part disinfectant system of claim 44, further comprising an alcohol.

79. The two-part disinfectant system of claim 78, wherein the alcohol includes a member selected from the group consisting of methanol, ethanol, a propanol, a butanol, a pentanol, and mixtures thereof.

80. The two-part disinfectant system of claim 78, wherein the alcohol includes a polyhydric alcohol.

81. The two-part disinfectant system of claim 44, adapted for admixture and application in the form of an aerosol, spray, pre-soaked fabric, gel, or foam.

82. The two-part disinfectant system of claim 44, in the form of a dilutable concentrate.

83. The two-part disinfectant system of claim 44, wherein the silver component is present in a form and at a concentration that causes silver staining when applied to a plastic surface, and the fluorescing compound and the silver ion fixing agent are present at concentrations that reduces the staining compared to an identical composition devoid of the fluorescing compound or the silver ion fixing agent.

84. The two-part disinfectant system of claim 44, further comprising an organic acid different than the silver ion fixing agent, a surfactant, a hypochlorite, a benzalkonium halide, an aldehyde, a chlorine-based disinfectant, a bromine-based disinfectant, an iodophore-based disinfectant, phenolic-based disinfectant, a quaternary ammonium-based disinfectant, a metal salt disinfectant other than silver salt, or combination thereof.

85. The two-part disinfectant system of claim 44, when admixed, being in the form of a dilutable concentrate.

86. The two-part disinfectant system of claim 44, when admixed, in the form of a ready to use solution or suspension.

87. The two-part disinfectant system of claim 44, wherein Part A comprises the fluorescing compound and the silver ion fixing agent.

88. The two-part disinfectant system of claim 44, wherein the silver ion fixing agent comprises both a chelating agent and a reducing agent.

89. A method of disinfecting a surface, comprising:
obtaining the system of claim 44;
combining Part A and Part B to form a reacting formulation; and
contacting the reacting formulation to the surface for a sufficient period of time to disinfect the surface.

90. The method of claim 89, further comprising the step of removing at least a portion of the composition from the surface after the surface is disinfected, but before any noticeable staining occurs on the surface.
